SEQUENCE LISTING (1 sequence)

SEQ ID NO: 1 - RNA, 50 nt, Unknown
  Description of Unknown recognition site of Robo1mRNA
uaucgaguuucauugcccagacaccggugucugggcaaugaaacucgaua

(19) United States
(12) Patent Application Publication
(10) Pub. No.: US 2020/0147233 A1
(43) Pub. Date: May 14, 2020

(54) METHOD FOR KILLING TUMOR CELLS AND COMPOSITION USED THEREFOR

(71) Applicant: PhotoQ3 Inc., Tokyo (JP)

(72) Inventors: Takao HAMAKUBO, Tokyo (JP); Noriko KOMATSU, Tokyo (JP); Kenichi MITSUI, Tokyo (JP); Osamu ARAI, Tokyo (JP); Hiroko IWANARI, Tochigi (JP); Tsuyoshi TAKATO, Tokyo (JP); Takahiro ABE, Saitama (JP)

(73) Assignee: PhotoQ3 Inc., Tokyo (JP)

(21) Appl. No.: 16/592,143

(22) Filed: Oct. 3, 2019

Related U.S. Application Data
(60) Provisional application No. 62/743,137, filed on Oct. 9, 2018.

(51) Int. Cl. A61K 47/68; A61K 41/00; A61P 35/00; A61N 5/06

(52) U.S. Cl. CPC A61K 47/6817; A61K 47/6849; A61K 47/6851; A61K 41/0071; A61N 2005/0659; A61N 5/062; A61N 2005/0651; A61N 2005/0654; A61N 2005/0662; A61P 35/00

(57) ABSTRACT

It is an object of the present invention to provide a method for killing tumor cells, having a few side effects. The present invention provides a method for killing tumor cells, comprising:
(1) a step of allowing an immunotoxin formed by binding an antibody binding to ROBO1 or a fragment thereof to a cytotoxin to come into contact with tumor cells;
(2) a step of allowing a photosensitizer that induces photochemical cytoplasmic internalization to come into contact with the tumor cells; and
(3) a step of irradiating the tumor cells with a wave length that is effective for activating the sensitizer, so as to kill the cells.

### TECHNICAL FIELD

The present invention relates to a method for killing tumor cells, comprising irradiating tumor cells with a light absorbed by a photosensitizer in the presence of the photosensitizer and a bound body of an antibody binding to ROBO1 and a cytotoxin. Further, the present invention relates to a bound body consisting of an antibody binding to ROBOT and a cytotoxin (ROBO1 antibody immunotoxin), which is used in the aforementioned method.

### BACKGROUND ART

Head and neck squamous cell carcinoma (HNSCC) is the 6th most common cancer in the world [Non-Patent Documents 1 and 2]. The incidence rate and the death rate of this cancer have almost unchanged for these 30 years [Non-Patent Document 3]. In addition to the death rate, another important issue of this disease is that postoperative complications caused by standard therapies such as surgery, chemotherapy and radiotherapy include nutritional disorder, speech disorder, and beauty problem, which lead to a significant decrease in the quality of life (QOL) in patients [Non-Patent Document 4]. Thus, it has been strongly desired to develop a novel therapy for reducing these treatment-related complications to the minimum. Monoclonal antibody therapy has been expected to satisfy these needs, but it has not yet exhibited sufficient effects on solid cancer.

Robo1 has first been discovered as an axon guidance receptor in Drosophila [Non-Patent Document 5]. The Robo family consists of Robo 1-4 [Non-Patent Document 6]. Human Robo1 has five immunoglobulin-like domains and three fibronectin III-like domains in the extracellular portion thereof [Non-Patent Document 6]. Robo1 as a cancer-specific antigen has been initially reported regarding liver cancer [Non-Patent Document 7]. However, at present, it has been known that Robo1 is expressed in a wide range of cancer types, such as colon cancer, breast cancer, pancreatic cancer, lung cancer, and head and neck squamous cell carcinoma [Non-Patent Documents 8 and 9]. It has been reported that Slit2/Robo1 signaling plays an important role in cancer infiltration, migration, epithelial-mesenchymal transition, cancer angiogenesis, and the like [Non-Patent Documents 8 and 9]. In recent years, Cetuximab and Nivolumab have been authorized as HNSCC treatment methods by the U.S. Food and Drug Administration (FDA) [Non-Patent Documents 10 and 11]. On the other hand, Trastuzumab-DM1 (Pertuzumab) has been authorized as a metastatic breast cancer treatment by FDA in 1998 [Non-Patent Document 12]. As a result of progression of a new-generation antibody-drug conjugate (ADC), the range of possible treatment has been widened, and a combination therapy with an immune checkpoint control therapy has entered clinical trials [Non-Patent Document 13].

### PRIOR ART DOCUMENTS

### Non-Patent Documents

Non-Patent Document 1: J. Ferlay, H. R. Shin, F. Bray, et al., Estimates of worldwide burden of cancer in 2008: GLOBOCAN 2008, Int. J. Cancer. 127 (2010) 2893-2917.
Non-Patent Document 2: A. Jemal, F. Bray, M. M. Center, et al., Global Cancer Statistics, CA CANCER J. CLIN. 61 (2011) 69-90.
Non-Patent Document 3: L. P. Chan, L. F. Wang, F. Y. Chiang, et al., IL-8 promotes HNSCC progression on CXCR1/2-mediated NOD1/RIP2 signaling pathway, Oncotarget. 7 (2016) 61820-61831.
Non-Patent Document 4: P. J. Thomson, J. Wylie, Interventional laser surgery: an effective surgical and diagnostic tool in oral precancer management, Int. J. Oral Maxillofac. Surg. 31 (2002) 145-153.
Non-Patent Document 5: T. Kidd, K. Brose, K. J. Mitchell, et al., Roundabout controls axon crossing of the CNS midline and defines a novel subfamily of evolutionarily conserved guidance receptors, Cell. 92 (1998) 205-215.
Non-Patent Document 6: M. S. Ballard, L. Hinck, A roundabout way to cancer, in: Ira O. Daar (Ed.), Advances in Cancer Research, Academic Press. 114 (2012) 187-235.
Non-Patent Document 7: H. Ito, S. Funahashi, N. Yamauchi et al., Identification of ROBO1 as a novel hepatocellular carcinoma antigen and a potential therapeutic and diagnostic target, Clin. Cancer Res. 12 (2006) 3257-3264.
Non-Patent Document 8: Y. Zhao, F. L. Zhou, W. P Li, et al., Slit2-robo1 signaling promotes the adhesion, invasion and migration of tongue carcinoma cells via upregulating matrix metalloproteinases 2 and 9, and downregulating E-cadherin, Molecular Medicine Reports. 14 (2016), 1901-1906.
Non-Patent Document 9: B. Wang, Y. Xiao, B.-B. Ding, et al., Induction of tumor angiogenesis by Slit-Robo signaling and inhibition of cancer growth by blocking Robo activity, Cancer Cell. 4 (2003) 19-29.
Non-Patent Document 10: M. A. Blasco, P. F. Svider, S. N. Raza, et al., Systemic therapy for head and neck squamous cell carcinoma: Historical perspectives and recent breakthroughs, Laryngoscope. (2017) 1-5.
Non-Patent Document 11: F. Zagouri, E. Terpos, E. Kastritis, et al., Emerging antibodies for the treatment of multiple myeloma, Expert Opin. Emerg. Drugs. 21 (2016) 225-37.
Non-Patent Document 12: I. Smith, M. Procter, R. D. Gelber, et al., 2-year follow-up of trastuzumab after adjuvant chemotherapy in HER2-positive breast cancer: a randomised controlled trial. Lancet. 369 (2007) 29-36.
Non-Patent Document 13: A. Thomas, B. A. Teicher, R. Hassan, Antibody-drug conjugates for cancer therapy, Lancet Oncol. 17 (2016) 254-262.
Non-Patent Document 14: K. Berg, A. Weyergang, L. Prasmickaite, et al., Photochemical Internalization (PCI): A Technology for Drug Delivery, Photodynamic Therapy MIMB. 635 (2010) 133-145.
Non-Patent Document 15: K. Berg, S. Nordstrand, P. K. Selbo, et al., Disulfonated tetraphenyl chlorin (TPCS2a), a novel photosensitizer developed for clinical utilization of photochemical internalization, Photochem. Photobiol. Sci. 10 (2011) 1637-51.
Non-Patent Document 16: K. Fujiwara, K. Koyama, K. Suga, et al., A 90Y-labelled anti-ROBO1 monoclonal antibody exhibits antitumour activity against hepatocellular carcinoma xenografts during ROBO1-targeted radioimmunotherapy, EJNMMI Res. 4 (2014)
Non-Patent Document 17: O. Kusano-Arai, R. Fukuda, W. Kamiya, et al., Kinetic exclusion assay of monoclonal antibody affinity to the membrane protein Roundabout 1 displayed on baculovirus, Analytical Biochemistry. 504 (2016) 41-49.
Non-Patent Document 18: O. Kusano-Arai, H. Iwanari, Y. Mochizuki, et al., Evaluation of the asparagine synthetase level in leukemia cells by monoclonal antibodies. Hybridoma (Larchmt). 31 (2012) 325-32.
Non-Patent Document 19: M. Shimizu, M. Imai, Effect of the antibody immunotherapy by the anti-MUC1 monoclonal antibody to the oral squamous cell carcinoma in vitro, Biol. Pharm. Bull. 31 (2008) 2288-93.
Non-Patent Document 20: G. P. Maiti, A. Ghosh, P. Mondal, et al., Frequent inactivation of SLIT2 and ROBO1 signaling in head and neck lesions: clinical and prognostic implications, Oral And Maxillofacial Pathology. 119 (2015), 202-212.
Non-Patent Document 21: W. J. Zhou, Z. H. Geng, S. Chi, et al., Slit-Robo signaling induces malignant transformation through Hakai-mediated E-cadherin degradation during colorectal epithelial cell carcinogenesis, Cell Research. 21 (2011) 609-626.
Non-Patent Document 22: S. Enomoto, K. Mitsui, T. Kawamura, et al., Suppression of Slit2/Robo1 mediated HUVEC migration by Robo4, Biochemical and Biophysical Research Communications. 469 (2015) 707-802.
Non-Patent Document 23: K. Fujiwara, K. Koyama, K. Suga, et al., 90Y-Labeled Anti-ROBO1 Monoclonal Antibody Exhibits Antitumor Activity against Small Cell Lung Cancer Xenografts, PLoS One. 10 (2015) 1-13.
Non-Patent Document 24: J. Meng, Y. Liu, S. Gao, et al., A bivalent recombinant immunotoxin with high potency against tumors with EGFR and EGFRvIII expression, Cancer Biology & Therapy. 16 (2015) 1764-1774.
Non-Patent Document 25: K. Berg, A. Dietze, O. Kaalhus, et al., Enhances the Antitumor Effect of Bleomycin, Clin. Cancer Res. 11 (2005) 8477-8485.
Non-Patent Document 26: M. Bostad, C. E. Olsen, Q. Peng, et al., Light-controlled endosomal escape of the novel CD133-targeting immunotoxin AC133-saporin by photochemical internalization—A minimally invasive cancer stem cell-targeting strategy, Journal of Control Release. 206 (2015) 37-48.

### SUMMARY OF INVENTION

### Object to be Solved by the Invention

It is an object of the present invention to provide a method for killing tumor cells, having a few side effects. It is another object of the present invention to provide an immunotoxin used in the above-described method for killing tumor cells.

Means for Solving the Object

Photochemical internalization (PCI) is a relatively new method, which is based on a photosensitizer and irradiation with a light having a wave length specific to the photosensitizer. When a photosensitizer is activated by light irradiation, singlet oxygen (¹O₂) is generated as a result of a photochemical reaction, and an endocytic membrane is destroyed [Non-Patent Document 14]. Since this method enables local irradiation, it is much more tumor-selective than ADC. At present, the present method using a new-generation photosensitizer, disulfonated tetraphenylchlorin (TPCS2a), has entered the phases I/II trial tests. In the present invention, the present inventors have used aluminum phthalocyanine disulfonic acid (AlPcS2a), which had been used as a photochemical drug delivery method, so far [Non-Patent Document 15]. In the present invention, the effects obtained by the combined use of an anti-Robo1 antibody saporin complex that is an immunotoxin targeting to Robo1 in HNSCC and PCI have been reported. The results of the present invention propose a novel antibody therapy involving PCI on HNSCC, and also suggest that the present invention expands the possibility of the treatment of cancer cell surface antigens with low expression levels as a drug delivery method.

According to the present invention, the following inventions are provided.

<1> A method for killing tumor cells, comprising:

(1) a step of allowing an immunotoxin formed by binding an antibody binding to ROBO1 or a fragment thereof to a cytotoxin to come into contact with tumor cells;

(2) a step of allowing a photosensitizer that induces photochemical cytoplasmic internalization to come into contact with the tumor cells; and

(3) a step of irradiating the tumor cells with a wave length that is effective for activating the sensitizer, so as to kill the cells.

<2> A method for killing tumor cells, comprising:

(1) a step of allowing an immunotoxin formed by binding an antibody binding to ROBO1 or a fragment thereof to cytotoxin and a photosensitizer that induces photochemical cytoplasmic internalization to come into contact with tumor cells; and then,

(2) a step of irradiating the tumor cells with a wave length that is effective for activating the sensitizer, so as to kill the cells.

<3> The method according to the above <1> or <2>, wherein the cytotoxin is saporin, gelonin, Pseudomonas Endotoxin, Shigatoxin, or a fragment or a genetically modified body thereof.
<4> The method according to the above <1> or <2>, wherein the photosensitizer is talaporfin sodium, aluminum phthalocyanine, or tetraphenylchlorin-2-sulfonic acid.
<5> The method according to any one of the above <1> to <4>, wherein the tumor cells express ROBO1 on the surface thereof.
<6> The method according to any one of the above <1> to <5>, wherein the tumor cells are cancer cells of any one of head and neck cancer, lung cancer, liver cancer, colon cancer, skin cancer, esophageal cancer, cervical cancer, pancreatic cancer, breast cancer, and osteosarcoma.
<7> A composition comprising an immunotoxin formed by binding an antibody binding to ROBO1 or a fragment thereof to cytotoxin, for use in killing tumor cells, wherein the composition kills tumor cells by the following steps:

(1) a step of allowing the immunotoxin to come into contact with tumor cells;

(2) a step of allowing a photosensitizer that induces photochemical cytoplasmic internalization to come into contact with the tumor cells; and

(3) a step of irradiating the tumor cells with a wave length that is effective for activating the sensitizer, so as to kill the cells.

<8> A composition comprising an immunotoxin formed by binding an antibody binding to ROBO1 or a fragment thereof to cytotoxin, for use in killing tumor cells, wherein the composition kills tumor cells by the following steps:

(1) a step of allowing an immunotoxin formed by binding an antibody binding to ROBO1 or a fragment thereof to cytotoxin and a photosensitizer that induces photochemical cytoplasmic internalization to come into contact with the tumor cells; and then,

(2) a step of irradiating the tumor cells with a wave length that is effective for activating the sensitizer, so as to kill the cells.

<9> The composition according to the above <7> or <8>, wherein the cytotoxin is saporin, gelonin, Pseudomonas Endotoxin, Shigatoxin, or a fragment or a genetically modified body thereof.
<10> The composition according to the above <7> or <8>, wherein the photosensitizer is talaporfin sodium, aluminum phthalocyanine, or tetraphenylchlorin-2-sulfonic acid.
<11> The composition according to any one of the above <7> to <10>, wherein the tumor cells express ROBO1 on the surface thereof.
<12> The composition according to any one of the above <7> to <11>, wherein the tumor cells are cancer cells of any one of head and neck cancer, lung cancer, liver cancer, colon cancer, skin cancer, esophageal cancer, cervical cancer, pancreatic cancer, breast cancer, and osteosarcoma.
<13> A kit for killing tumor cells, comprising:

(1) an immunotoxin formed by binding an antibody binding to ROBO1 or a fragment thereof to cytotoxin tumor cells, for use in killing tumor cells; and

(2) a photosensitizer that induces photochemical cytoplasmic internalization.

### Advantageous Effects of Invention

According to the present invention, a method for killing tumor cells, which has a few side effects, can be provided.

### BRIEF DESCRIPTION OF DRAWINGS

FIG. 1 shows the expression level of Robo1 in various HNSCC cancer cells. (a and b) The Robo1 protein bands were detected in HSQ-89 and Sa3, and both of them were reduced by a specific siRNA treatment. (c) Evaluation of the expression level of Robo1 on the cell surface according to a flow cytometric analysis. MFI: mean fluorescence intensity (arbitrary unit).

FIG. 2 shows the results obtained by examining the enhancement of the cytotoxic activity of anti-Robo1 antibody immunotoxin (IT-Rpbp1) according to photochemical internalization (PCI). (a) The cytotoxic activity of IT-Robo1 on Robo1/CHO is Robo1-specific and dose-dependent. (b) The cytotoxic activity of IT-Robo1. (c) The cytotoxic activity of IT-Robo1. (d) The significant dose-dependent effects of PCI observed in HSQ-89 cells. IC₅₀ is approximately 34 pM.

FIG. 3 shows the results obtained by examining that the cell line Sa3 expressing a low level of Robo1 exhibits reactivity to IT and PCI as a result of long-term irradiation. (a and b) IT-Robo1 (c) Cytotoxic activity on Sa3. (d) A significant cytotoxic activity was not observed in SAS.

FIG. 4 shows the results obtained by examining the antitumor effects of the combined use of IT-Robo1 and PCI (AlPcS₂ₐ, 650 nm LED) on HSQ-89 cell line xenograft mice. (a) Change in the size of a tumor, and (b) change in body weight.

FIG. 5 shows the results obtained by performing a cytotoxicity test, in which IT-Robo1 was used in combination with PCI (AlPcS₂ₐ, 650 nm LED) in the HSQ-89 cell line xenograft mice (photographs of mice on Day 14 after administration of IT-Robo1). (1) IT-Robo1+AlPcS₂ₐ combined use group, (2) IT-Robo1 single administration group, (3) AlPcS₂ₐ single administration group, and (4) control group, wherein the black arrow indicates a tumor site.

### EMBODIMENT OF CARRYING OUT THE INVENTION

Hereinafter, the embodiments of the present invention will be described in detail.

### <Abbreviations>

Head and neck squamous cell carcinoma: HNSCC
Quality of life: QOL

### Immunotoxin: IT

Antibody drug conjugate: ADC

### U.S. Food and Drug Administration: FDA

Photochemical internalization: PCI
Chinese hamster ovary cells: CHO

### SUMMARY OF PRESENT INVENTION

Head and neck squamous cell carcinoma (HNSCC) is the 6th most common cancer in the world. Postoperative complications caused by standard therapies such as surgery, chemotherapy and radiotherapy include nutritional disorder, speech disorder, beauty problem, which lead to a significant decrease in the quality of life (QOL) in patients. In recent years, an antibody drug has been increasingly recognized as a novel treatment method for enhancing such QOL. Robo1 as a nerve axon guidance receptor has attracted much attention as a target of such an antibody drug in various types of cancer species.

The present inventors have examined the expression level of Robo1 in HNSCC cell lines and have also examined the effects of an immunotoxin (IT) formed by adding saporin to an anti-Robo1 antibody. The expression level of Robo1 on the cell surface was evaluated by a flow cytometric method using the anti-Robo1 antibody B5209B. As a result, it was found that approximately 220,000 copies were expressed in a single cell in the case of CHO cells forcibly expressing Robo1 (Robo1/CHO); 22,000 copies were expressed in a single cell in the case of HSQ-89 cell line (HNSCC); 3,000 copies were expressed in a single cell in the case of Sa3 cell line, and almost no expression was found in SAS cell line. IT treatment as a conventional method did not show insufficient cytotoxic effects even on HSQ-89 cells. However, if light irradiation (650 nm) with a photochemical sensitizer and LED was added to the IT treatment, significant cytotoxic effects were found on HSQ-89 cells. Also in Sa3 cells, cytotoxic effects were found by prolonging the light irradiation time.

From these results, it is considered that photochemical internalization (PCI) is effective as a means for enhancing the tumor-killing effects of IT. Since the drug delivery method shown in the present studies can also be applied to other target molecules of low abandance on the surface of cancer cells, it would expand the possibility of developing new drugs used towards cancers, for which a treatment method has not yet been developed.

### <Embodiments Regarding Method for Killing Cells>

The method for killing tumor cells of the present invention includes an embodiment in which tumor cells are irradiated with (3) a light having a wave length for activating (2) a photosensitizer that induces photochemical internalization in the coexistence of (1) a ROBO1 immunotoxin and the photosensitizer.

As a further specific embodiment, tumor cells can also be killed by administering (1) a ROBOT immunotoxin to a subject, then administering thereto (2) a photosensitizer that induces photochemical internalization, and then irradiating the cells with (3) a light having a wave length for activating the photosensitizer.

In addition, as another embodiment, tumor cells can also be killed by administering (1) a photosensitizer that induces photochemical internalization to a subject, then administering (2) a ROBO1 immunotoxin thereto, and then irradiating the cells with (3) a light having a wave length for activating the photosensitizer.

Moreover, as another embodiment, tumor cells can also be killed by simultaneously administering (1) a ROBO1 immunotoxin and (2) a photosensitizer to a subject, and then irradiating the cells with (3) a light having a wave length for activating the photosensitizer.

### <Photosensitizer>

The photosensitizer used in the present invention is a sensitizer that induces photochemical internalization as a result of the activation thereof with a light, and is preferably a sensitizer that generates singlet oxygen as a result of the activation thereof with a light.

Examples of the photosensitizer used in the present invention may include a photosensitizer and a photosensitive substance, which are proposed or used for PCI (Photochemical Internalization) or PDT (Photodynamic Therapy).

Specific examples of the photosensitizer used in the present invention may include tetraphenylchlorin-2-sulfonic acid (TPCS2a) and a salt thereof, and disulfonated aluminum phthalocyanine (AlPcS2 and AlPcS2a) and a salt thereof.

Moreover, other examples may include sulfonated tetraphenylporphyrin (e.g., TPPS₂ₐ, TPPS₄, TPPS₁, and TPPS₂₀), nile blue, a chlorin derivative, bacteriochlorin, ketochlorin, and natural and synthetic porphyrin.

Furthermore, other specific examples may include talaporfin sodium (LASERPHYRIN®), porfimer sodium (PHOTOFRIN®), 5-aminolevulinic acid (Levulan®), and 5-amino levulin methyl ester (Metvix®).

Further specific examples may include protoporphyrin IX, foscan, chlorin, uroporphyrin I, uroporphyrin III, heptacarboxylporphyrin I, heptacarboxylporphyrin III, hexacarboxylporphyrin I, hexacarboxylporphyrin III, pentacarboxylporphyrin I, pentacarboxylporphyrin III, coproporphyrin I, coproporphyrin III, isocoproporphyrin, harderoporphyrin, isoharderoporphyrin, hematoporphyrin, mesoporphyrin, ethioporphyrin, pyroporphyrin, deuteroporphyrin IX, pemptoporphyrin, ATXs-10, and a 5-aminolevulinic acid derivative.

The photosensitizer used in the present invention may be activated by absorbing a visible light, but it is also preferable for the present sensitizer to absorb a visible light with a long wave length or a near infrared light.

Examples of such a sensitizer may include silicon phthalocyanine, zinc phthalocyanine, and a derivative thereof. Further specific examples may include IR700® and a derivative thereof.

### <ROBO1 Antibody>

The Robo1 antibody immunotoxin used in the present invention is formed by binding an antibody binding to ROBOT or a fragment of the antibody with a cytotoxin.

The type of the antibody used in the present invention is not particularly limited, and examples of the present antibody may include a mouse antibody, a human antibody, a rat antibody, a rabbit antibody, a sheep antibody, a camel antibody, an avian antibody, and a genetically modified antibody that is artificially modified for the purpose of reducing xenoantigenicity against a human, such as a chimeric antibody or a humanized antibody. Such a genetically modified antibody can be produced by applying a known method. The chimeric antibody is an antibody consisting of the heavy chain and light chain variable regions of a mammalian antibody other than a human antibody, such as a mouse antibody, and the heavy chain and light chain constant regions of a human antibody. The chimeric antibody can be obtained by ligating DNA encoding the variable region of a mouse antibody to DNA encoding the constant region of a human antibody, then incorporating the ligate into an expression vector, and then introducing the expression vector into a host, so that the host is allowed to generate the antibody. The humanized antibody is obtained by transplanting the complementarity determining region (CDR) of a mammalian antibody other than a human antibody, such as a mouse antibody, into the complementarity determining region of a human antibody. A common gene recombination method therefor has been known. Specifically, a DNA sequence designed to ligate the CDR of a mouse antibody to the framework region (FR) of a human antibody is synthesized from several oligonucleotides that have been produced such that they have an overlapping portion at the terminal portions thereof according to a PCR method. The obtained DNA is ligated to DNA encoding the constant region of a human antibody, and the ligate is then incorporated into an expression vector, which is then introduced into a host, so that the host is allowed to generate the antibody (EP 239400, International Publication WO96/02576, etc.).

In addition, a method for obtaining a human antibody has also been known. For example, human lymphocytes are sensitized with a desired antigen or a cell expressing the desired antigen in vitro, and then fusing the sensitized lymphocytes with human myeloma cells, such as, for example, U266, so as to obtain a desired human antibody having a binding activity to an antigen (JP Paten Publication (Kokoku) No. 1-59878 B (1989)). Otherwise, a transgenic antibody having all repertoires of human antibody genes is immunized with a desired antigen to obtain a desired human antibody (see WO93/12227, WO92/03918, WO94/02602, WO94/25585, WO96/34096, and WO96/33735). Further, a technique of obtaining a human antibody by panning using a human antibody library has also been known. For example, a human antibody variable region is allowed to express as a single chain antibody (scFv) on the surface of a phage according to a phage display method, and a phage binding to an antigen can be then selected. By analyzing the selected phage gene, a DNA sequence encoding the variable region of a human antibody binding to the antigen can be determined. If the DNA sequence of scFv binding to an antigen is clarified, a suitable expression vector comprising the sequence can be produced, so that a human antibody can be obtained. These methods have already been publicly known, and please refer to WO92/01047, WO92/20791, WO93/06213, WO93/11236, WO93/19172, WO95/01438, and WO95/15388.

The antibody binding to ROBO1 is preferably a humanized or a human antibody, but is not limited thereto.

Moreover, these antibodies may also be low molecular weight antibodies such as antibody fragments, or modified forms of the antibodies, unless they lose the property of recognizing the entire or a part of a protein encoded by a ROBO1 gene. The antibody fragment is a part of an antibody that retains a binding ability to ROBO1. Specific examples of the antibody fragment may include Fab, Fab′, F(ab′)2, Fv, Diabody, and a single chain variable fragment (scFv). In order to obtain such an antibody fragment, a gene encoding such an antibody fragment is constructed, the gene is then introduced into an expression vector, and it may be then expressed in suitable host cells. As a modified form of an antibody, an antibody binding to various types of molecules such as polyethylene glycol (PEG) can also be used.

DNA encoding a monoclonal antibody can be easily isolated and sequenced according to a commonly used method (for example, by using an oligonucleotide probe capable of specifically binding to a gene encoding the heavy chain and light chain of the monoclonal antibody). Hybridoma cells may be preferable starting materials for such DNA. Once such DNA is isolated, it is inserted into an expression vector, and the expression vector is then used to transform host cells such as E. coli cells, COS cells, CHO cells, or myeloma cells that do not generate immunoglobulin before they are transformed. Then, a monoclonal antibody can be generated from the transformed host cells.

An example of the ROBO1 antibody may be the monoclonal antibody B5209B described in JP Patent Publication (Kokai) No. 2008-290996 A and International Publication WO2010/131590. Hybridoma that generates the monoclonal antibody B5209B was deposited with the National Institute of Advanced Industrial Science and Technology (AIST), International Patent Organism Depositary (IPOD), (Higashi 1-1-1, Center 6, Tsukuba-shi, Ibaraki-ken, Japan, postal code: 305-8566), under Accession No. FERM P-21238 on Mar. 2, 2007. Thereafter, this strain was transferred to an international deposition under Accession No. FERM BP-10921 on Oct. 16, 2007.

### <Cytotoxin>

The cytotoxin binding to an antibody is preferably a protein having cytotoxicity, but is not limited thereto. The cytotoxin may also be a low molecular weight compound having a synthetic or natural anticancer action.

Preferred examples of such a protein having cytotoxicity may include saporin, gelonin, Pseudomonas Endotoxin, ricin A chain, deglycosylated ricin A chain, a ribosome inactivating protein, alphasarcine, aspergillin, restrictocin, ribonuclease, epipodophyllotoxin, diphtheria toxin, Shigatoxin, and a fragment, a mutant or a genetically modified body thereof.

### <ROBO1 Antibody Immunotoxin>

The Robo1 antibody immunotoxin, the antibody binding to ROBO1 or a fragment of the antibody, and the cytotoxin must directly or indirectly bind to one another.

As a method of directly chemically binding the antibody or a fragment thereof to the cytotoxin, a binding method used for known ADC (Antibody Drug Conjugate; antibody-enzyme complex) can be used. Otherwise, when the cytotoxin is a protein, a bifunctional crosslinking agent can also be used.

Alternatively, when the cytotoxin is a protein, toxin is fused with an antibody or a fragment thereof by genetic recombination to form a protein, so that an immunotoxin can be produced.

Moreover, as another method, a method of indirectly binding an antibody or a fragment thereof to a cytotoxin by using a second binding pair can also be used. Examples of the second binding pair that can be utilized herein may include avidin-biotin and an antibody-hapten.

### <Target Cells/Diseases>

The tumor as a target of the present invention includes all tumors that express ROBO1 on the surface thereof, and the target tumor preferably expresses 1,000 or more ROBO1 molecules.

Specific examples of the target tumor may include cancers such as head and neck cancer, lung cancer, liver cancer, colon cancer, skin cancer, esophageal cancer, cervical cancer, pancreatic cancer, breast cancer, and osteosarcoma.

In addition, ROBO1 is also expressed in neovascular endothelial cells. Thus, by killing such neovascular cells, disease can be treated. Examples of the disease that is treated by killing neovascular cells may include macular degeneration, rheumatoid arthritis, and cancer.

The present invention is specifically explained with reference to the following Examples below; however, the present invention is not limited to the Examples.

### EXAMPLES

### Example 1

### (1) Materials and Methods

### <Cell Culture>

The HNSCC cell lines, namely, HSQ-89 (derived from maxillary sinus; RCB0789), HO-1-u-1 (derived from floor of mouth; RCB2012), Sa3 (derived from upper jaw; RCB0980), and SAS (derived from tongue; RCB1974) were acquired from RIKEN, Institute of Physical and Chemical Research (Saitama, Japan). HSQ-89 cells were cultured in a Dulbecco's modified Eagle's medium (DMEM) supplemented with 10% fetal bovine serum. HO-1-u-1 and SAS cells were cultured in an RPMI1640 medium supplemented with 10% fetal bovine serum. Sa3 cells were cultured in a basic minimum essential medium (BMEM) supplemented with 20% fetal bovine serum. CHO cells (CH-K1 and CCL-61) were acquired from American Type Culture Collection (ATCC) (Bethesda, Md.). CHO cells forcibly overexpressing Robo1 (Robo1/CHO) and CHO cells forcibly overexpressing Robo4 (Robo4/CHO) were established as mentioned above, using a Flp-In gene expression system (Thermo Fisher Scientific, Massachusetts) [16]. The Robo1/CHO and Robo4/CHO cells were cultured in a HamF12 medium supplemented with 10% fetal bovine serum.

### <Reverse Transcription Real-Time PCR>

Using RNeasy Plus Mini Kit (QIAGEN, Germany), total RNA was extracted from the cultured cells. To examine the expression level of mRNA, desired mRNA was reversely transcribed by using SuperScript™ III First-Strand Synthesis System (Thermo Fisher Scientific, Massachusetts) to synthesize cDNA, and the cDNA was then subjected to quantitative PCR using SYBR Green PCR master mix (Takara, Japan).

The expression level was standardized with glyceraldehyde-3-phosphate dehydrogenase (GAPDH) RNA, and it was defined to be the relative expression level of Robo1 mRNA.

### <Western Blot Analysis>

A protein mixture (cell lysate) (7.5 μg per lane, but 0.75 μg only in the case of Robo1/CHO) was separated according to SDS-PAGE using 10% acrylamide-containing gel, and was then transferred on a nitrocellulose membrane (GE Healthcare Life Science, U.K.). The membrane was treated with Block Ace (DS Pharma, Osaka) for 1 hour, and was then reacted with the anti-Robo1 antibody A7241A (1.0 μg/ml, an antibody established in our laboratory) used as a primary antibody for 2 hours [Non-Patent Document 7]. Subsequently, the resultant was reacted with a secondary antibody that was a peroxidase conjugated anti-mouse IgG goat antibody (Jackson, Me.)(5,000-fold diluted) had been added. The Robo1 band on the membrane was subjected to chemical luminescence, using Super-signal West Dura Extended Duration Substrate (Thermo Fisher Scientific, Massachusetts), and was then detected using ImageQuant LAS 500 (GE Healthcare Life Science, U.K.).

### <RNA Interference>

In accordance with an instruction manual included with Lipofectamine RNAiMAX Reagent (Thermo Fisher Scientific, Massachusetts), an RNA interference experiment was carried out. The cells were seeded at a cell density of 5×10³ cells/well on a 6-well plate, 200 nM each siRNA, together with RNAiMAX Reagent, was added to each well. After completion of the reaction for 40 hours, the expression level of Robo1 was analyzed by a Western blot method. The recognition site of Robo1mRNA is as follows:

5′-UAUCGAGUUUCAUUGCCCAGACACCGGUGUCUGGGCAAUGAAACUCGA
UA-3′.

As a negative control, Stealth RNAi Negative Control Kit (Thermo Fisher Scientific, Massachusetts) was used (hereinafter referred to as “si-Control”).

### <Flow Cytometry>

The cells were seeded at a cell density of 1×10⁶ cells/well on a 96-well plate, and were then centrifuged at 2,000 rpm at 4° C. for 2 minutes, and a supernatant was then removed. The precipitated cells were reacted with the anti-Robo1 monoclonal antibody B5209B (10 μg/ml) (produced in our laboratory) [Non-Patent Documents 16 and 17], or with a negative control that was 100 μl of a sorting buffer (phosphate buffered saline (PBS) containing 1% bovine serum albumin, 0.1 mM ethylenediaminetetraacetic acid (EDTA), and 0.1% Proclin 300) containing 1 μg of Hyb3423 (10 μg/ml), on ice for 60 minutes. Thereafter, the resultant was washed with a sorting buffer. The resulting cells were further reacted with R-Phycoerythin AffiniPure F(ab′)2 Fragment Goat Anti Mouse IgG antibody (Jackson, Me.) on ice for 60 minutes. The resultant was further washed with a sorting buffer twice, and was then analyzed using Guava EasyCyte plus flow cytometer (Merck, Germany).

The amount of the Robo 1 antigen on the cell surface (antigen molecules/cell) was quantified from the histogram of calibration beads of QIFKIT (Agilent Technologies, California), as mentioned previously in [Non-Patent Document 18].

### <Immunotoxin Cytotoxicity Assay>

Hereinafter, the saporin-added anti-Robo1 antibody (B5209B) and the saporin-added negative control antibody (B8109B) are referred to as “IT-Robo1” and “IT-NC,” respectively. These antibodies were reacted with 2 μl of 1.1 μM biotinylated antibody and 2 μl of 1.1 μM streptavidin-saporin (Biotin-Z Internalization Kit [KIT-27-Z], Advanced Targeting Systems, California) at room temperature for 30 minutes, and 46 μl of a cell culture medium was then added to each reaction mixture, so that the concentration of IT was adjusted to 42 nM. The Robo1/CHO cells and the HSQ-89 cells were each seeded at a cell density of 2.5×10³ cells/well on a 96-well plate, and were then cultured overnight. Thereafter, the cultured cells were reacted with various concentrations (0.0013 to 4.2 nM) of IT-Robo 1 or IT-NC for 72 hours (FIG. 2a) or 168 hours (FIG. 2b), respectively.

A CCK-8 kit solution (Cell Counting Kit-8, DOJINDO LABORATORIES, Japan) was added in an amount of 10 μl/well to the cells, so that they were reacted for 40 minutes to 2 hours, and then, the absorbance at 450 nm was measured. Thereafter, the cell survival percentage was calculated according to an instruction manual included with the kit.

The cell survival percentage is calculated according to the following calculating equation:

Cell survival percentage (%)=(a−c)/(b−c)×100.

In the above equation, “a” indicates the absorbance of each specimen, “b” indicates the absorbance of a negative control specimen not containing IT, and “c” indicates the absorbance of a blank containing only a medium [Non-Patent Document 19]. From three independent experiments, the mean value±SD of cell survival percentages was obtained, and it was plotted to the IT concentration on a graph. Regarding the IC50 value, using the Nonlinear Generalized Reduced Gradient method (GRG) of the excel software, a sigmoid curve was fitted to the mean value plot on the graph, so as to obtain the IT concentration showing 50% survival rate.

### <Cytotoxicity Assay According to PCI>

As a photosensitizer, aluminum phthalocyanine disulfonic acid AlPcS₂ₐ having a sulfonic acid group on the phthalic acid ring thereof was purchased from Frontier Scientific (Utah), and was then used. An LED lamp (54 W) having a peak wave length at 650 nm was purchased from King Do Way (18PCS E27, Amazon.co.jp).

Various types of cells were seeded at a cell density of 2.5×10³ cells (Robo1/CHO), 5.0×10³ cells (SAS) or 2.0×10⁴ cells (HSQ-89 and Sa3) per well on a 96-well plate, and were then cultured at 37° C. overnight. The optimal concentration of AlPcS2a was determined for each of various types of cell lines. Based on the results, the concentration of AlPcS2a was determined to be 5.0 μg/ml for Robo1/CHO, HSQ-89 and Sa3, and was determined to be 0.5 μg/ml for SAS, and it was then used in a cytotoxicity assay. Together with the photosensitizer, IT-Robo 1 or IT-NC immunotoxin was added to each well to a final concentration of 0.0013 to 4.2 nM, and the obtained mixture was then incubated for 16 hours. Thereafter, the culture solution was replaced with a culture solution to which no drugs had been added, and the culture was further continued for 4 hours. Subsequently, the obtained culture was irradiated with the LED lamp for 5 minutes (62.7 mW/cm2, 18.8 J/cm2). Seventy-two hours later, the cell survival percentage was measured using the CCK-8 kit mentioned in the section “Immunotoxincytotoxicity assay.” For Sa3 and SAS, long-term irradiation (10 minutes) was carried out (62.7 mW/cm2, 37.6 J/cm2). The IC50 value was calculated as described above in the section “Immunotoxincytotoxicity assay.”

### <Statistical Processing>

The data are shown with the mean value±SD. After completion of analysis of variance (ANOVA), statistical evaluation was carried out by the Tukey Honest Significant Differences test. The p value<0.01 was defined to be statistically significant.

### (2) Results

### <Expression Level of Robo1 in Various HNSCC Cancer Cells>

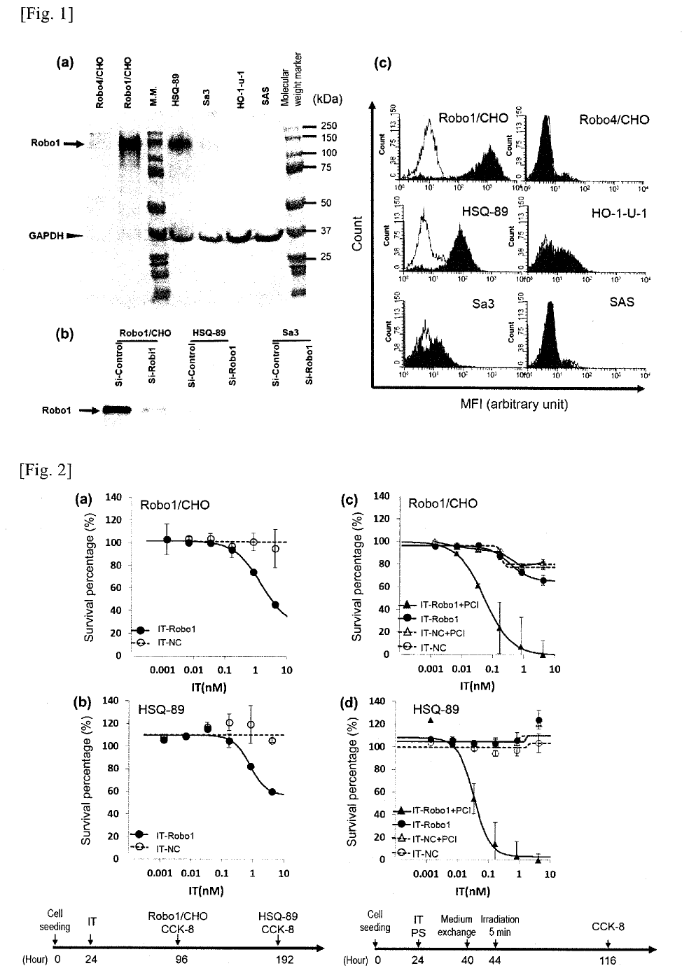

As described in the section “Materials and Methods,” the expression level of Robo1 mRNA was examined according to qPCR. The amount of a Robo1 protein on each cell surface was evaluated according to Western blot or flow cytometry, using the anti-Robo1-specific antibodies A7241A and B5209B, respectively. In the HSQ-89 and Sa3 cells, the Robo1 protein band was found around approximately 200 k daltons, and was reduced by a siRNA treatment in both cases (FIGS. 1a and 1b). The expression level of the Robo1 protein was well correlated with the level of mRNA in various types of cell lines (Table 1). With regard to the expression level of Robo1 on the cell surface according to flow cytometry (FIG. 1c), it was estimated that approximately 220,000 copies were expressed in a single cell in the case of Robo1/CHO cells, approximately 22,000 copies were expressed in a single cell in the case of HSQ89 cells, approximately 3,000 copies were expressed in a single cell in the case of Sa3 cells, and approximately 200 copies were expressed in a single cell in the case of HO-1-u-1 cells. Further, in the case of SAS cells, an extremely small number of copies were found (Table 1). As shown in Table 1, the expression level of Robo 1 was well correlated with the protein and the mRNA in various types of cell lines.

TABLE 1

| | Robo1 mRNA | | Robo1 protein | |
|---|---|---|---|---|
| Cell line | (GAPDH relative ratio) | | (copies/cell) | |
| Robo1/CHO | 1.97 ± | 0.798 | 220,000 ± | 15,800 |
| HSQ-89 | 0.281 ± | 0.389 | 22,300 ± | 4,900 |
| Sa3 | 0.0226 ± | 0.00687 | 3,010 ± | 138 |

| | | | |
|---|---|---|---|
| HO-1-u-1 | ND | 184 ± | 80.6 |
| SAS | ND | 33.7 ± | 67.0 |

| | | |
|---|---|---|
| Robo4/CHO | ND | ND |

Indicated with a mean value ± SD of three independent experimental values.
ND: Not detectable

### <Enhancement of Cytotoxic Activity of Anti-Robo 1 Antibody Immunotoxin (IT-Robo 1) According to Photochemical Internalization (PCI)>

The cytotoxic activity of IT-Robo1 on Robo1/CHO cells was specific to Robo 1 and was dose-dependent (FIG. 2a). However, the effects were only 60% even in the highest concentration applied (4.2 nM) (FIG. 2a). These results demonstrate that internalization of IT-Robo1 was insufficient. The cytotoxic activity of IT-Robo1 on HSQ-89 cells was weaker, and it was 40% even in the highest concentration applied (FIG. 2b).

For the purpose of increasing IT-Robo 1 internalization efficiency, the present inventors have used AlPcS2a, which forms reactive oxygen species as a result of light irradiation and induces destruction of endosome. At first, the cytotoxicity of AlPcS2a itself was examined, and the optimal concentration to various cells was then determined. Except for SAS cells, all types of cells exhibited almost equivalent dose-dependent AlPcS2a resistance. SAS cells are weak to the treatment with a photosensitizer, and thus, the present inventors conducted PCI experiments, using a 0.5 μg/ml photosensitizer for SAS cells, and a 5.0 μg/ml photosensitizer for other types of cells.

AlPcS2a was added to the culture solution, and sixteen hours later, the reaction mixture was irradiated with 650 nm LED for 5 minutes. As a result, IT-Robo 1 exhibited sufficient cytotoxic activity on Robo1/CHO cells. This cytotoxic activity was dose-dependent effect (which was significant in ANOVA), and IC50 was approximately 54 pM (FIG. 2c). Such dose-dependent cytotoxic activity was significantly observed in HSQ-89 cells, and IC50 was approximately 34 pM (FIG. 2d).

### <Cell Line Sa3 Expressing Low Level of Robo1 Exhibits Reactivity to IT and PCI by Long-Term Irradiation>

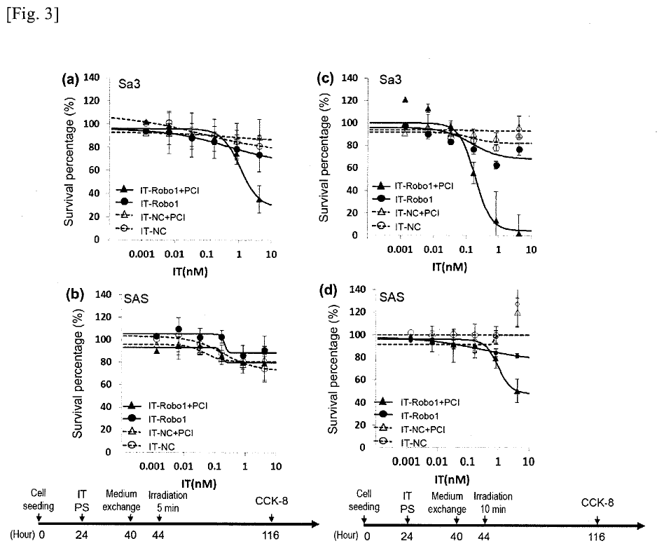

The expression level of Robo 1 was low in Sa3 and SAS cells, and according to the calculation by flow cytometry, approximately 3,000 and 30 copies were expressed per cell, respectively (Table 1). In these cells, the effects of IT-Robo1 were not found as a result of irradiation for 5 minutes (FIGS. 3a and 3b). However, when the light irradiation time was prolonged to 10 minutes and the energy was increased by 2 times, significant dose-dependent cytotoxic activity was apparently found in Sa3 cells (FIG. 3c) (ANOVA and Tukey HSD post analysis test, p=0.00061). In the case of SAS cells, cytotoxic activity was not found even after the prolonged light irradiation (FIG. 3d) (ANOVA, p=0.0196).

### (3) Discussion

Maiti et al. have reported that, in head and neck cancer, the expression level of Robo 1 mRNA was reduced, whereas the expression level of the Robo 1 protein was increased at a middle to high level [Non-Patent Document 20]. Zhao et al. have reported that Slit2/Robo 1 signaling promotes the adhesion, infiltration and migration of tongue cancer cells via the inhibitory control of E cadherin [Non-Patent Document 8]. Zhou et al. have suggested similar inhibitory effects of Slit/Robo1 signaling on epithelial cell cancer of the colon and rectum [Non-Patent Document 21]. Hence, it is considered that when Robo1 is highly expressed in a certain organ, the organ can be a good target of antibody therapy.

As a result of the research conducted by the present inventors, the expression level of Robo1 mRNA was well correlated with the expression level of the protein. It is considered that a difference between the present results and the results of Maiti et al. was dependent on the specimens used [Non-Patent Document 20]. In other cell lines, such as, for example, HepG2 [Non-Patent Document 7], HUVEC [Non-Patent Document 22], and squamous cell lung cancer [Non-Patent Document 23], the correlated expression of the mRNA and the protein was shown.

The effects of new-generation ADC on solid cancer were significantly improved [Non-Patent Document 13]. Thus, the present inventors examined application of a toxin-added anti-Robo 1 antibody to oral cancer. An immunotoxin formed by adding saporin to B5209B that was an antibody having high affinity for Robo1 (Kd=30 pM) [Non-Patent Documents 16 and 17] exhibited insufficient cytotoxic activity even on CHO cells forcibly overexpressing Robo1 (Robo1/CHO cells) (FIGS. 2a and 2b). These results suggest that cellular internalization of IT-Robo 1 was insufficient. Hence, the present inventors attempted to use the photosensitizer AlPcS2a that had been reported to generate singlet oxygen as a result of light irradiation and to promote destruction of the endosome. Red light (650 nm LED, 62.7 mW/cm2, 18.8 J/cm2) was applied for 5 minutes to HSQ-89 cells expressing 20,000 Robo1 copies per cell, and as a result, sufficient cytotoxic effects were observed (IC50=0.034 nM) (FIG. 2). The IC50 value of EGFR- and EGFRvIII-specific divalent recombinant IT (DT390-BiscFv806) to various HNSCC cell lines is 0.24 nM to 156 nM [Non-Patent Document 24]. Thus, it is suggested that the IT-Robo 1 by AlPcS2a according to the present study is sufficient effective for clinical application.

Furthermore, attention should also be paid to the fact that effective cytotoxic activity on Sa3 cells can be obtained by prolonging the irradiation time to 10 minutes. Because only approximately 3,000 Robo1 copies are expressed per cell in this cell line, and this result suggests that the IT-PCI therapy can be applied to a wide range of HNSCC cases, even to precancerous lesions (dysplasia) [20] in which the expression of the Robo1 protein is reportedly at a low to middle level.

### (4) Conclusion

It has been reported that the combined use of the anticancer agent bleomycin and PCI exhibits synergic effects on two types of tumor models [Non-Patent Document 25]. Bostad et al. have reported that immunotoxin AC133-saporin that targets the cancer stem cell marker CD133 has effective cytotoxic activity only when the cancer cells are treated by PCI [Non-Patent Document 26]. As such, the combined use of immunotoxin AC133-saporin with PCI provides the possibility of a locally effective, minimally invasive cancer treatment method.

The present study demonstrated that the combined use of PCI and IT can provide strong and sufficient cytotoxic effects on cancer cells, on which the single use of IT is not effective because of insufficient internalization. Moreover, the present study also demonstrated that such cytotoxic effects are increased by prolonging the light irradiation time, and suggested that the combined use of PCI and IT can also be applied to other cancer cell surface targets with low expression levels. From the aforementioned results, it is anticipated that the therapeutic method will be expanded to refractory cancers.

### Example 2: Concerning Tumor-Reducing Effect of Combined Use of Saporin-Added Anti-Robo1 Antibody (Immunotoxin IT-Robo1) and PCI on Cancer-Bearing Mice

### (1) Methods and Materials

### <Cell Culture>

Cell culture was carried out in the same manner as that in Example 1.

HSQ-89 cell line (derived from maxillary sinus squamous cell carcinoma)
Culture solution: DMEM, 10% FBS, and 90 units/ml penicillin/90 μg/ml streptomycin
Production of saporin-added anti-Robot antibody immunotoxin (IT-Robot) (same as that in Example 1)
PCI (photosensitizer; same as that in Example 1) AlPcS₂ₐ was used.

### <Production of HSQ-89 Cell Line Xenograft Mice>

A supernatant was aspirated from a 10 cmφ dish, in which the HSQ-89 cell line was cultured, and was then washed with D-PBS (Dulbecco's phosphate buffer). Thereafter, 1 ml of 2.5 g/l trypsin/1 mM EDTA solution was added to the resultant, and the obtained mixture was then left at rest for several minutes in a CO₂ incubator. After confirming that the cells were peeled from the bottom surface of the dish, 9 ml of the culture solution was added, and centrifugation was then performed at 1,000 rpm at 4° C. for 5 minutes. After that, a supernatant was aspirated, 10 ml of PBS was then added thereto, and the obtained mixture was then centrifuged at 1,000 rpm at 4° C. for 5 minutes. Thereafter, a supernatant was aspirated, 10 ml of PBS was added thereto, and the number of cells was then counted. Again, 10 ml of PBS was added to the cells, and the obtained mixture was then centrifuged at 1,000 rpm at 4° C. for 5 minutes. Thereafter, a supernatant was aspirated, and PBS was added thereto to a cell density of 2×10⁷ cell/ml. Basement Membrane Matrix Gel (Corning, N.Y.) that had been cooled on ice was added in an equal amount, and 2×10⁶ cells/200 μl were then collected using a 23G needle and a 1-ml syringe that had been cooled on ice. Subsequently, the collected cells were subcutaneously administered (SC) to the right shoulder of 6-weel-old male BALB/cSlc-nu/nu mice to produce xenograft mice (cancer-bearing mice). The size of a tumor was calculated according to the following equation:

Tumor size=π/6×Height (mm)×Width (mm)×Depth (mm)

### <Analysis Regarding Antitumor Effects of Combined Use of IT-Robo1 and PCI on HSQ-89 Cell Line Xenograft Mice>

When the size of a mouse tumor reached 40 mm³, the mice were randomly divided into the following groups (1) to (4), and administration was carried out at n=5. IT-Robo1 or D-PBS was administered to the mice, and three days after the administration, AlPcS₂ₐ was administered as PCI to the mice, or D-PBS was administered as a control thereto. Thirty minutes later, red LED with a wave length of 650 nm was locally applied to the tumor portion for 30 minutes (62.7 mW/cm², 113 J/cm²). A margin of 2 to 3 mm was established around the tumor portion, and the sites other than the tumor portion were covered with a thick brown paper.

(1) IT-Robo1+AlPcS₂ₐ combined use group: intraperitoneal administration of 16 μg/200 IT-Robo1 (B5209B-biotin+streptavidin-saporin)+subcutaneous administration of 100 μg/100 μl AlPcS2a
(2) IT-Robo 1 single administration group: intraperitoneal administration of 16 μg/200 μl IT-Robo1+subcutaneous administration of 100 μI of D-PBS
(3) AlPcS₂ₐ single administration group: intraperitoneal administration of 200 μl of D-PBS+subcutaneous administration of 100 μg/100 μl AlPcS₂ₐ
(4) control group: intraperitoneal administration of 200 μl of D-PBS+subcutaneous administration of 100 μl of D-PBS

With reference to “Guidelines for Proper Conduct of Animal Experiments (2006)” established by Science Council of Japan, a tumor with a size of 1,000 mm³ or more, or a drastic reduction in body weight (a reduction of 25% or more of body weight for a week) was set at endpoint, and the mice were euthanized by cervical dislocation. The measurement of a tumor size and evaluation of the body weight were carried out over time, and at the endpoint, the size and weight of the tumor and the body weight were evaluated.

### <Antitumor Effects of Combined Use of IT-Robo1 and PCI on HSQ-89 Cell Line Xenograft Mice>

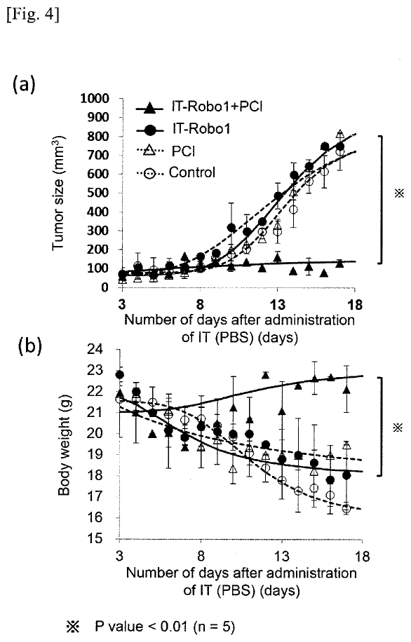

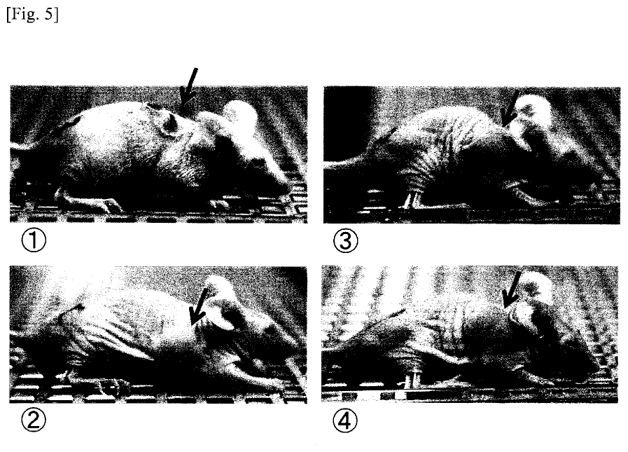

The antitumor effects of the combined use of IT-Robo1 and PCI on the HSQ-89 cell line xenograft mice were analyzed. Significant suppression of the tumor enlargement was observed in (1) IT-Robo1+AlPcS₂ₐ combined use group, in comparison to (2) IT-Robo1 single administration group, (3) AlPcS₂ₐ single administration group, and (4) control group (ANOVA analysis, p<0.01) (FIG. 4(a)). In addition, a reduction in the body weight was significantly suppressed in (1) IT-Robo1+AlPcS₂ₐ combined use group, in comparison to (2) IT-Robo1 single administration group, (3) AlPcS₂ₐ single administration group, and (4) control group (ANOVA analysis, p<0.01) (FIG. 4(b)). The photographs of mice 14 days after initiation of the treatment (administration of IT-Robo1) are shown in FIG. 5. In (1) IT-Robo1+AlPcS₂ₐ combined use group, a retardation in the tumor enlargement was confirmed even by visual observation, in comparison to (2) IT-Robo 1 single administration group, (3) AlPcS₂ₐ single administration group, and (4) control group (FIG. 5).

Moreover, in (1) IT-Robo 1+AlPcS₂ₐ combined use group and (3) AlPcS₂ₐ single administration group, as a result of the local irradiation of the cancerous portions with 650 nm, edema was found at the irradiated sites and the surrounding portions thereof for several days from the next day, but such edema was gradually reduced. Furthermore, an ulcer was formed at the cancer protrusion site in the range in which such edema was generated, about two days after the irradiation, and thereafter, a crust was formed (FIG. 5(1)). Such a crust fell off 10 days after the crust formation in both (1) IT-Robo 1+AlPcS₂ₐ combined use group and (3) AlPcS₂ₐ single administration group. Dissection was carried out at the stage of requiring euthanasia. In (1) IT-Robo 1+AlPcS₂ₐ combined use group and (3) AlPcS₂ₐ single administration group, the site in which an ulcer and a crust were formed tended to adhere to the epithelium, and thus, the site tended to be slightly hardly removed, in comparison to (2) IT-Robo1 single administration group and (4) control group.

1. A method for killing tumor cells, comprising:
(1) allowing an immunotoxin formed by binding an antibody binding to ROBO1 or a fragment thereof to a cytotoxin to come into contact with tumor cells;
(2) allowing a photosensitizer that induces photochemical cytoplasmic internalization to come into contact with the tumor cells; and
(3) irradiating the tumor cells with a wave length that is effective for activating the sensitizer, so as to kill the cells.

2. A method for killing tumor cells, comprising:
(1) allowing an immunotoxin formed by binding an antibody binding to ROBO1 or a fragment thereof to cytotoxin and a photosensitizer that induces photochemical cytoplasmic internalization to come into contact with tumor cells; and then,
(2) irradiating the tumor cells with a wave length that is effective for activating the sensitizer, so as to kill the cells.

3. The method according to claim 1, wherein the cytotoxin is saporin, gelonin, Pseudomonas Endotoxin, Shigatoxin, or a fragment or a genetically modified body thereof.

4. The method according to claim 1, wherein the photosensitizer is talaporfin sodium, aluminum phthalocyanine, or tetraphenylchlorin-2-sulfonic acid.

5. The method according to claim 1, wherein the tumor cells express ROBO1 on the surface thereof.

6. The method according to claim 1, wherein the tumor cells are cancer cells of any one of head and neck cancer, lung cancer, liver cancer, colon cancer, skin cancer, esophageal cancer, cervical cancer, pancreatic cancer, breast cancer, and osteosarcoma.

7. A composition comprising an immunotoxin formed by binding an antibody binding to ROBO1 or a fragment thereof to cytotoxin, for use in killing tumor cells, wherein the composition kills tumor cells by the following steps:
(1) allowing the immunotoxin to come into contact with tumor cells;
(2) allowing a photosensitizer that induces photochemical cytoplasmic internalization to come into contact with the tumor cells; and
(3) irradiating the tumor cells with a wave length that is effective for activating the sensitizer, so as to kill the cells.

8. A composition comprising an immunotoxin formed by binding an antibody binding to ROBO1 or a fragment thereof to cytotoxin, for use in killing tumor cells, wherein the composition kills tumor cells by the following steps:
(1) allowing an immunotoxin formed by binding an antibody binding to ROBO1 or a fragment thereof to cytotoxin and a photosensitizer that induces photochemical cytoplasmic internalization to come into contact with the tumor cells; and then,
(2) irradiating the tumor cells with a wave length that is effective for activating the sensitizer, so as to kill the cells.

9. The composition according to claim 7, wherein the cytotoxin is saporin, gelonin, Pseudomonas Endotoxin, Shigatoxin, or a fragment or a genetically modified body thereof.

10. The composition according to claim 7, wherein the photosensitizer is talaporfin sodium, aluminum phthalocyanine, or tetraphenylchlorin-2-sulfonic acid.

11. The composition according to claim 7, wherein the tumor cells express ROBO1 on the surface thereof.

12. The composition according to claim 7, wherein the tumor cells are cancer cells of any one of head and neck cancer, lung cancer, liver cancer, colon cancer, skin cancer, esophageal cancer, cervical cancer, pancreatic cancer, breast cancer, and osteosarcoma.

13. A kit for killing tumor cells, comprising:
(1) an immunotoxin formed by binding an antibody binding to ROBO1 or a fragment thereof to cytotoxin tumor cells, for use in killing tumor cells; and
(2) a photosensitizer that induces photochemical cytoplasmic internalization.
